(19) United States
(12) Patent Application Publication
(10) Pub. No.: US 2026/0193715 A1
(43) Pub. Date: Jul. 9, 2026

(54) SYSTEMS AND METHODS FOR IDENTIFYING CLONAL EXPANSION OF ABNORMAL LYMPHOCYTES

(71) Applicant: GRAIL, Inc., Menlo Park, CA (US)

(72) Inventors: Jing XIANG, San Carlos, CA (US); Qinwen LIU, Fremont, CA (US); Oliver Claude VENN, San Francisco, CA (US); Samuel S. GROSS, Sunnyvale, CA (US)

(73) Assignee: GRAIL, Inc., Menlo Park, CA (US)

(21) Appl. No.: 19/130,467

(22) PCT Filed: Nov. 15, 2023
(86) PCT No.: PCT/US2023/079859

§ 371 Date: May 15, 2025

Related U.S. Application Data
(60) Provisional application No. 63/425,907, filed on Nov. 16, 2022.

(51) Int. Cl. C12Q 1/6886; G16B 30/00; G16B 40/20

(52) U.S. Cl. CPC C12Q 1/6886; G16B 30/00; G16B 40/20

(57) ABSTRACT

Systems and methods for determining a disease state of a subject are disclosed. One method may include: determining a disease state of a subject by conducting one or more biological assays analyzing a biological sample of the subject; responsive to determining that the subject has the positive disease state, generating an immune repertoire profile of the subject, the immune repertoire profile generated from an immune repertoire sequencing of the biological sample and comprising a plurality of clonotypes and corresponding clonal frequencies of the clonotypes; identifying one or more clonal expansions of one or more clonotypes in the immune repertoire profile; determining, based on the one or more clonal expansions, that the subject is associated with a heme condition; and determining, based on the determined heme condition and the disease state determined by the one or more biological assays, that the positive disease state is a false positive.

### CROSS REFERENCE TO RELATED APPLICATIONS

This application claims the benefit of priority to U.S. Provisional Application No. 63/425,907, filed on Nov. 16, 2022, which is incorporated by reference herein in its entirety.

### TECHNICAL FIELD

The present disclosure relates generally to systems and methods for distinguishing blood conditions from cancer in individuals and, more specifically, to the use of companion diagnostic testing to enhance the accuracy of cancer detection.

### BACKGROUND

Accurately distinguishing blood conditions from cancer in individuals may be challenging. This challenge may be exacerbated by the presence of confounding signals from white blood cells (WBCs) in diagnostic samples, leading to false positives. Additionally, precursor hematological conditions, such as Monoclonal B-cell Lymphocytosis (MBL) and Monoclonal Gammopathy of Undetermined Significance (MGUS), may exhibit DNA methylation patterns resembling cancer, further complicating detection. One or more aspects of this disclosure may address one or more of the issues described above.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, systems and methods are described for leveraging a companion diagnostic test, in association with a disease state classifier, to determine whether a subject has a heme condition that may affect the results of the disease state classifier.

In summary, one aspect provides a method for determining a heme condition of a subject from a biological sample of the subject. The method may include: receiving an immune repertoire profile of the subject, the immune repertoire profile generated from an immune repertoire sequencing of deoxyribonucleic acid (DNA) in the biological sample and comprising a plurality of clonotypes of the DNA and corresponding clonal frequencies of the clonotypes; identifying one or more clonal expansions of one or more clonotypes in the immune repertoire profile; inputting the clonal frequencies associated with the one or more clonal expansions to a machine learning model that is iteratively trained based on training samples, the training samples comprising immune repertoire profiles of reference individuals with known disease states, the disease states comprising a first disease state where no heme condition is diagnosed, a second disease state where the heme condition is diagnosed, and a third disease state where a cancer is diagnosed, wherein the reference individuals in the training samples comprise individuals with one or more of the disease states; and generating, using the machine learning model, a determination of the heme condition of the subject.

In another aspect, a method for determining a disease state of a subject is disclosed. The method may include: determining a disease state of a subject by conducting one or more biological assays analyzing a biological sample of the subject; responsive to determining that the subject has the positive disease state, generating an immune repertoire profile of the subject, the immune repertoire profile generated from an immune repertoire sequencing of the biological sample and comprising a plurality of clonotypes and corresponding clonal frequencies of the clonotypes; identifying one or more clonal expansions of one or more clonotypes in the immune repertoire profile; determining, based on the one or more clonal expansions, whether the subject is associated with a heme condition; and determining, responsive to determining that the subject is associated with the heme condition and based on the disease state determined by the one or more biological assays, that the positive disease state is a false positive.

In yet another aspect, a system is disclosed. The system may include: one or more processors; one or more computer readable media storing instructions that are executable by the one or more process to perform operations to: determine a disease state of a subject by conducting one or more biological assays; generate, responsive to determining that the subject has the positive disease state, an immune repertoire profile of the subject, the immune repertoire profile generated from an immune repertoire sequencing of the biological sample and comprising a plurality of clonotypes and corresponding clonal frequencies of the clonotypes; identify one or more clonal expansions of one or more clonotypes in the immune repertoire profile; determine, based on the one or more clonal expansions, whether the subject is associated with a heme condition; and determine, responsive to determining that the subject is associated with the heme condition and based on the disease state determined by the one or more biological assays, that the positive disease state is a false positive.

In yet another aspect, a non-transitory computer-readable medium storing computer-executable instructions is disclosed. The computer-executable instructions may cause the system to perform operations including: determining a disease state of a subject by conducting one or more biological assays analyzing a biological sample of the subject; generating, responsive to determining that the subject has the positive disease state, an immune repertoire profile of the subject, the immune repertoire profile generated from an immune repertoire sequencing of the biological sample and comprising a plurality of clonotypes and corresponding clonal frequencies of the clonotypes; identifying one or more clonal expansions of one or more clonotypes in the immune repertoire profile; determining, based on the one or more clonal expansions, whether the subject is associated with a heme condition; and determining, responsive to determining that the subject is associated with the heme condition and based the disease state determined by the one or more biological assays, that the positive disease state is a false positive.

In yet another aspect, a system is disclosed. The system may include: one or more processors; one or more computer readable media storing instructions that are executable by the one or more process to perform operations to: determine a disease state of a subject by conducting one or more biological assays; generate, responsive to determining that the subject has the positive disease state, an immune repertoire profile of the subject, the immune repertoire profile generated from an immune repertoire sequencing of the biological sample and comprising a plurality of clonotypes and corresponding clonal frequencies of the clonotypes; identify one or more clonal expansions of one or more clonotypes in the immune repertoire profile; determine, based on the one or more clonal expansions, whether the subject is associated with a heme condition; and determine, responsive to determining whether the subject is associated with the heme condition and based on the disease state determined by the one or more biological assays, whether the positive disease state is a false positive.

In yet another aspect, a non-transitory computer-readable medium storing computer-executable instructions is disclosed. The computer-executable instructions may cause the system to perform operations including: determining a disease state of a subject by conducting one or more biological assays analyzing a biological sample of the subject; generating, responsive to determining that the subject has the positive disease state, an immune repertoire profile of the subject, the immune repertoire profile generated from an immune repertoire sequencing of the biological sample and comprising a plurality of clonotypes and corresponding clonal frequencies of the clonotypes; identifying one or more clonal expansions of one or more clonotypes in the immune repertoire profile; determining, based on the one or more clonal expansions, whether the subject is associated with a heme condition; and determining, responsive to determining whether the subject is associated with the heme condition and based on the disease state determined by the one or more biological assays, whether the positive disease state is a false positive.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate several embodiments and together with the description, serve to explain the principles of the disclosure.

FIG. 1A depicts an exemplary computer system for executing the methods described herein.

FIG. 1B depicts an exemplary software platform for executing the methods described herein.

FIG. 2 depicts an exemplary workflow for utilizing a companion test to validate a disease state determination from a disease state classifier, according to one or more embodiments of the present disclosure.

FIG. 3 depicts an exemplary graph illustrating observed data for subjects having the precursor heme condition MBL, according to one or more embodiments of the present disclosure.

FIG. 4 depicts another exemplary graph illustrating observed data for subjects having the precursor heme condition MGUS, according to one or more embodiments of the present disclosure.

FIG. 5 depicts another exemplary graph illustrating observed data for solid cancer false positives, according to one or more embodiments of the present disclosure.

FIG. 6 depicts another exemplary graph illustrating data associated with a group of participants that had leukemia, according to one or more embodiments of the present disclosure.

FIG. 7 depicts another exemplary graph illustrating data associated with a group of participants that had multiple myeloma (MM), according to one or more embodiments of the present disclosure.

FIG. 8 depicts another exemplary graph depicting data associated with a distribution of negative controls, according to one or more embodiments of the present disclosure.

FIG. 9 depicts an exemplary diagram, according to one or more embodiments of the present disclosure.

FIG. 10 depicts an exemplary graph depicting a histogram of cancer scores for subjects, according to one or more embodiments of the present disclosure.

FIG. 11 depicts an example computing system, according to one or more embodiments of the present disclosure.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of +10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “user” generally encompasses any person or entity, such as a researcher and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

One challenge in the realm of blood-based diagnostic tests is the emergence of confounding signals originating from WBCs. WBCs play an important role in the body's immune response, and their presence in diagnostic samples can introduce complicating factors that affect the interpretation of test results. These confounding signals originating from an immune system response, can occasionally lead to misleading outcomes. Consequently, they can create an obstacle in the accurate detection of cancer and precursor conditions.

Another challenge revolves around the phenomenon of clonal expansion in precursor hematological conditions, such as MBL and MGUS. These conditions are of particular concern, especially among older populations, as they may exhibit methylation patterns in their DNA that bear a resemblance to those found in cancer. Moreover, abnormal lymphocytes, encompassing both lymphoid and myeloid cells, can release their DNA into the bloodstream. This has the potential to confound circulating cell-free DNA tests used in cancer detection, as these samples might be erroneously classified as cancer, leading to false positives and undermining the overall sensitivity and accuracy of the tests.

Diagnostic tests have been developed to detect cancer and precursor conditions. For example, the DETECT-A test from THRIVE has been used for baseline cancer testing. A confirmation test component utilizes DNA from WBCs to exclude Clonal Hematopoiesis of Indeterminate Potential (CHIP) mutations and is performed for participants with a positive baseline test. However, the DETECT-A test is limited in that it focuses on a targeted single nucleotide variant (SNV) panel to detect CHIP mutations associated with myeloid cells, thereby excluding the broader range of precursor conditions.

Accordingly, the present disclosure is designed to address one or more of the foregoing challenges by providing a companion diagnostic test for identifying whether a subject is associated with a heme condition that may affect the results of a disease state assay. To facilitate this process, in an aspect, a disease state of a subject may be determined by analyzing a biological sample of a subject via a biological assay (e.g., a targeted methylation assay). In this regard, the biological sample may be collected, genomic DNA may be collected and sequenced, and the sequenced data may be processed and subsequently provided to a machine learning model that is trained to identify whether methylation patterns in the genomic DNA sample correlate to known methylation patterns associated with certain disease states, e.g., certain cancers. Upon receiving an indication from the classifier that the subject has tested positive for a disease state, an immune repertoire profile may be generated for the subject. The immune repertoire profile may be generated via an immune repertoire sequencing technique and may include a plurality of clonotypes and corresponding clonal frequencies of the clonotypes. In an aspect, one or more clonal expansions of the one or more clonotypes in the immune repertoire profile may be identified. Thereafter, the identified clonal expansions may be utilized to determine whether the subject is associated with a particular heme condition. Responsive to determining that the subject is associated with a particular heme condition, a system of the embodiments may correspondingly determine that the positive disease state determination by the disease state classifier is a false positive.

The concepts described herein may overcome at least some of the limitations of prior diagnostic methods by introducing a novel companion diagnostic test that is not confined to specific gene mutations or myeloid cells into a disease state determination workflow. More particularly, the companion test described herein does not rely on a targeted SNV panel limited to specific genes, thereby allowing for the detection of abnormal clonal lymphocytes originating from both lymphoid and myeloid cells. Additionally, unlike tests limited to detecting CHIP mutations within certain genes, the companion test may identify precursor conditions of both myeloid and lymphoid origin.

The development of the companion diagnostic test described herein involves the integration of various technologies, such as immune repertoire sequencing assays, machine learning algorithms, and specific DNA sequencing techniques, to identify and quantify abnormal clonal lymphocytes. More particularly, the application incorporates machine learning algorithms as part of the diagnostic test, which serves to enhance the diagnostic process by allowing the system to learn and adapt based on data patterns. This integration improves the efficiency and accuracy of the diagnostic test over time, making it a more intelligent and adaptive computer-based system. Specifically, by utilizing advanced computation methods, the companion test aims to provide a more nuanced and accurate assessment, reducing false positives and enhancing overall diagnostic precision. Furthermore, the concepts described herein also address a real-world problem in the medical diagnostic field by accurately detecting and differentiating precursor heme conditions from cancerous conditions. This practical application distinguishes the concepts described herein from other techniques by providing a tangible benefit in the field of healthcare. Additionally, the processes executed by the computer involve complex calculations and data manipulations on a large amount of biological data that a human individual could not reasonably complete on their own or in their mind. Specifically, computationally intensive statistical tests are leveraged by the computer to evaluate differences between sample sets, processes which cannot be completed by a human user.

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments,” or “in one aspect” or “in some aspects” as used herein does not necessarily refer to the same embodiment or aspect, and the phrase “in another embodiment” or “in another aspect” as used herein does not necessarily refer to a different embodiment or aspect. It is intended, for example, that claimed subject matter include combinations of exemplary embodiments in whole or in part.

It is also important to note that although the concepts described throughout this disclosure are made in reference to cancer, these designations are for exemplary purposes only and are not intended to be limiting. Specifically, the concepts described herein may be applicable to other disease types and other disease-detecting classifiers. More generally, the companion test described herein may be configured to provide a probability that an individual testing positive for a disease state (e.g., other than cancer) may harbor a precursor blood condition that contributes to a false positive disease state.

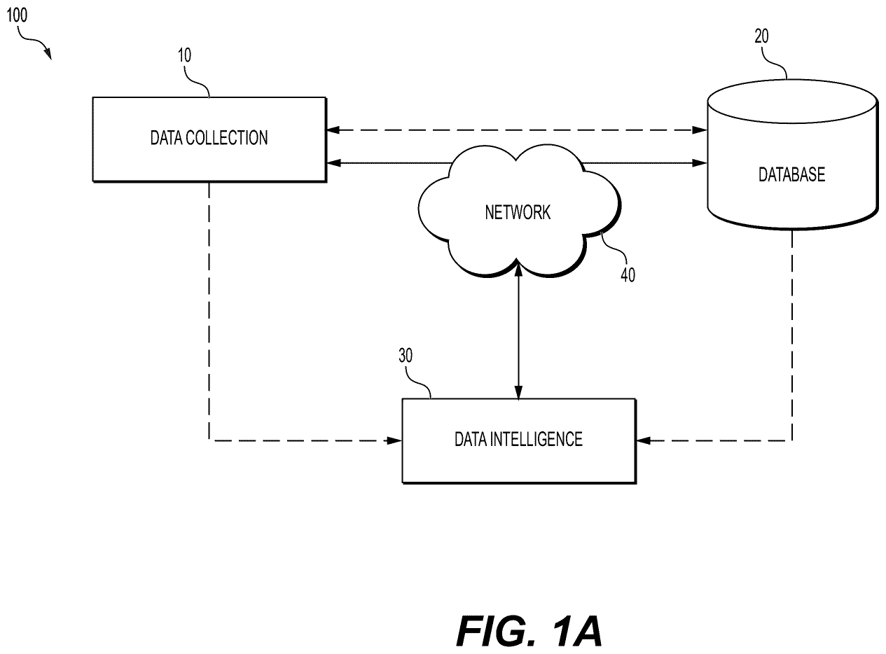

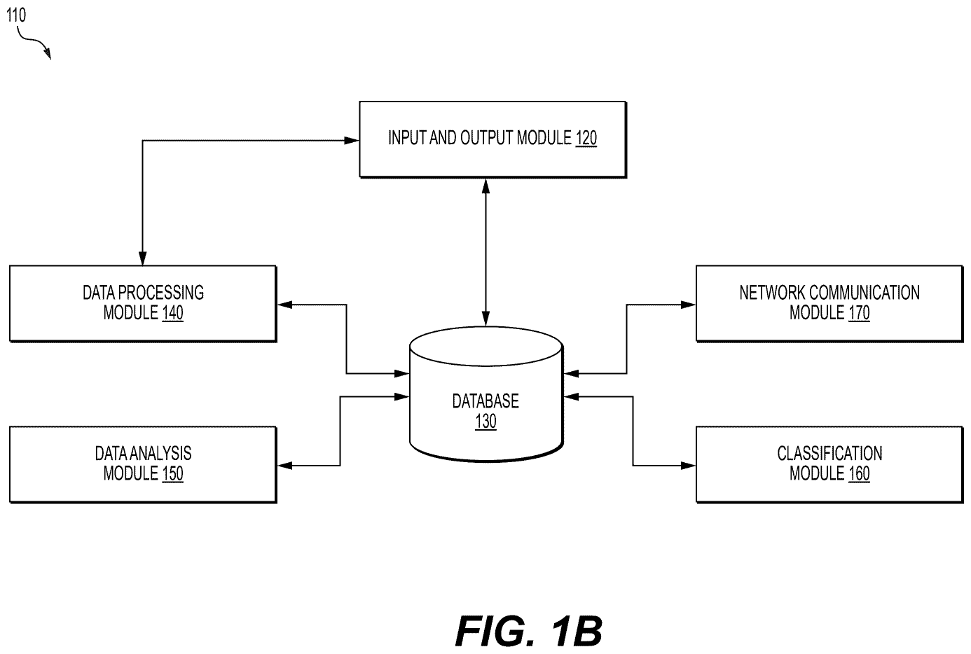

FIG. 1A depicts an exemplary system for utilizing a companion test in conjunction with a disease state classifier. Exemplary system 100 includes a data collection component 10, a database 20, and device data intelligence component 30, operably connected to each other via network 40. Alternatively, or additionally, one or more of the components may be connected with another component locally without reliance on network connection; e.g., through a wired connection. In many aspects described herein, sequencing data of cell-free nucleic acids are used to illustrate the concepts. However, one of skill in the art would understand that the current method may be applied to sequencing data of DNA, RNA, or other materials, as well from a variety of sample types, e.g., a blood sample (e.g., a serum sample, a plasma sample, a whole blood sample), a urine sample, a saliva sample, a tissue sample, a bone marrow sample, etc.

As disclosed herein, data collection component 10 may include a device or machine with which sequencing data may be generated. In some embodiments, data collection component 10 may include one or more sequencing devices or a facility that uses one or more sequencing devices to generate nucleic acid (e.g., DNA or RNA) sequence data of biological samples. In some aspects, data collection 10 may be a database that receives sequencing information generated from one or more sequencing devices. Any suitable liquid or solid biological samples may be used for sequencing. In some embodiments, a biological sample may be cell-based, for example, one or more types of tissue. In some embodiments, a biological sample may be a sample that includes cell-free nucleic acid fragments. Examples of biological samples include, but are not limited to, a blood sample (e.g., cfDNA sample, a genomic DNA (gDNA) sample, a serum sample, a plasma sample, a whole blood sample, a buffy coat sample, etc.), a urine sample, a saliva sample, a tissue sample, a bone marrow sample, etc. Further, although sequencing of DNA from these samples is discussed herein, RNA from these samples may alternatively or additionally be sequenced.

Examples of sequencing data may include, but are not limited to, sequence read data of targeted genomic locations, partial or whole genome sequencing data of the genome represented by nucleic acid fragments in cell-free or cell-based samples, partial or whole genome sequencing data including one or more types of epigenetic modifications (e.g., methylation), or combinations thereof.

Data acquired by the data collection component 10 may be transferred to database 20 via network 40 or a local or network connection. In some embodiments, data collection component 10 may alternatively receive data from one or more sequencing devices. In some embodiments, the collected data may be analyzed by data intelligence component 30, via network 40 or a local or network connection. FIG. 1B depicts exemplary functional modules that may be implemented to perform tasks of data intelligence component 30.

FIG. 1B depicts an exemplary computer system 110 for utilizing a companion test in conjunction with a trained disease state classifier. Exemplary system 110 achieves such functionalities by implementing, on one or more computer devices, user input and output (I/O) module 120, memory or database 130, data processing module 140, data analysis module 150, classification module 160, network communication module 170, and any other functional modules that may be needed for carrying out a particular task (e.g., an error correction or compensation module, a data compression module, etc.). As disclosed herein, user I/O module 120 may further include an input sub-module, such as a keyboard, and an output sub-module, such as a display (e.g., a printer, a monitor, or a touchpad). In some embodiments, all functionalities may be performed by one computer system. In some embodiments, the functionalities are performed by more than one computer system. The various modules (e.g., for data processing, analysis, classification, communication, etc.) may be one or more processes executing in a distributed computing environment. For instance, in some embodiments, one or more components of the computer system 110 may be network accessible via cloud infrastructure. For example, the database 130 used to store data may be stored in one or more remote cloud servers. In this regard, the database may be one or more large storage buckets (e.g., cloud-based storage buckets such as simple storage service “S3” buckets, etc.) from which data may be retrieved on demand. As another example, data processing, analysis, and classification may be performed in cloud-based environments using services like cloud-based data processing platforms, serverless computing, cloud-based machine learning platforms, and the like.

Also disclosed herein, a particular task may be performed by implementing one or more functional modules. In particular, each of the enumerated modules itself may, in turn, include multiple sub-modules. For example, data processing module 140 may include a sub-module for data quality evaluation (e.g., for discarding very short sequence reads or sequence reads including obvious errors), a sub-module for normalizing numbers of sequence reads that align to different regions of a reference genome, a sub-module to compensate/correct GC biases, a sub-module for matching data associated with a cancer sample with other data associated with one or more non-cancer samples, etc.

In some embodiments, a user may use I/O module 120 to manipulate data that is available either on a local device or can be obtained via a network connection from a remote service device or another user device. For example, I/O module 120 may allow a user, e.g., via a keyboard, a mouse, or a touchpad, to perform data analysis via a graphical user interface (GUI). In some embodiments, a user may manipulate data via voice control. In some embodiments, user authentication may be required before a user is granted access to the data being requested. In some embodiments, user I/O module 120 may be used to manage various functional modules. For example, a user may request via user I/O module 120 input data while an existing data processing session is in process. A user may do so by selecting a menu option or type in a command discretely without interrupting the existing process. In another example, a user may utilize user I/O module 120 to set various thresholds, configure sample matching settings, and/or provide other instructions to computer system 110 that dictate how results from the companion test or an associated classifier are processed, stored, and/or utilized. As disclosed herein, a user may use any type of input to direct and control data processing and analysis via I/O module 120.

In some embodiments, system 110 further comprises a memory or database 130. In some embodiments, database 130 comprises a local database that may be accessed via user I/O module 120. In some embodiments, database 130 comprises a remote database that may be accessed by user I/O module 120 via network connection. In some embodiments, database 130 is a local database that stores data retrieved from another device (e.g., a user device or a server). In some embodiments, memory or database 130 may store data retrieved in real-time from internet searches. In some embodiments, database 130 may send data to and receive data from one or more of the other functional modules, including, but not limited to, a data collection module (not shown), data processing module 140, data analysis module 150, classification module 160, network communication module 170, and etc. In some embodiments, some or all of the sample data may be stored on database 130.

In some embodiments, database 130 may be a database local to the other functional modules. In some embodiments, database 130 may be a remote database that may be accessed by the other functional modules via wired or wireless network connection (e.g., via network communication module 170). In some embodiments, database 130 may include a local portion and a remote portion.

In some embodiments, system 110 comprises a data processing module 140. Data processing module 140 may receive data from I/O module 120 or database 130. In some embodiments, data processing module 140 may perform standard data processing algorithms, such as one or more of noise reduction, signal enhancement, normalization of counts of sequence reads, correction of GC bias, etc. In some embodiments, data processing module 140 may be configured to identify features in DNA methylation data. For example, computer system 110 may be able to identify one or more differentially methylated regions (DMRs), which are regions where DNA methylation varies significantly between different biological samples.

In some embodiments, system 110 comprises a data analysis module 150. In some embodiments, data analysis module 150 includes instructions for identifying and treating systematic errors in sequencing data, as described in connection with data processing module 140.

In some embodiments, system 110 comprises a classification module 160, which may embody a “machine-learning model” or “trained classifier.” As used herein, a “machine-learning model” or “trained classifier” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as k-nearest neighbors, linear regression, logistic regression, random forest, gradient boosted machine (GBM), deep learning, a deep neural network, decision trees, support vectors, and/or any other suitable machine-learning technique that solves problems in the field of Natural Language Processing (NLP). Supervised, semi-supervised, and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

In an exemplary use case, a machine-learning model may be trained to analyze data from a test sample from a test subject whose status with respect to a medical condition is unknown and subsequently classifies the unknown test sample from the test subject based on the likelihood of the subject fitting into a particular category. In some embodiments, the one or more parameters may include a score (e.g., a binomial probability score that is calculated based on logistic regression analysis). As disclosed herein, the score may correspond to the likelihood of a subject having a certain medical condition, such as cancer. For example, a score of over a predefined threshold may indicate that the subject associated with a test sample is more likely to have cancer than not have cancer. Additionally or alternatively, in another aspect, the score may correspond to the likelihood of a subject having a heme condition and/or that a previous determination of a positive disease state is indicative of a false positive. In some embodiments, the one or more parameters may include a sequencing or methylation data distribution pattern correlating with the presence of cancer. A subject associated with a test sample having sequencing or methylation data with a pattern resembling the cancer pattern may be diagnosed as having cancer. In some embodiments, a sequencing or methylation data distribution pattern may be identified in connection with a specific type of cancer, thus allowing a test sample to be classified as indicative of a certain cancer type.

In some aspects, the foregoing score may be associated with a methylation sequencing pipeline in which biological samples are collected and bisulfite conversion is implemented to prepare cfDNA. Subsequent high-throughput sequencing, data preprocessing, and methylation calling may be conducted to identify methylated and unmethylated CpG sites. Differential methylation analysis may pinpoint cancer-associated regions, and feature selection processes may extract relevant CpG sites. A trained machine learning model may be configured to analyze the relevant features and generate a score (e.g., a cancer score) that represents the likelihood of disease presence. A thresholding process may be employed to categorize samples into minimal residual disease (MRD) positive or MRD-negative categories. This integrated pipeline may help support clinical decisions by providing a quantitative MRD assessment based on methylation data.

As disclosed herein, network communication module 170 may be used to facilitate communications between a user device, one or more databases, and any other suitable system or device through a wired or wireless network connection. Any communication protocol/device may be used, including, without limitation, a modem, an Ethernet connection, a network card (wireless or wired), an infrared communication device, a wireless communication device, and/or a chipset (such as a Bluetooth™ device, an 802.11 device, a WiFi device, a WiMax device, cellular communication facilities, etc.), a near-field communication (NFC), a Zigbee communication, a radio frequency (RF) or radio-frequency identification (RFID) communication, a PLC protocol, a 3G/4G/5G/LTE based communication, and/or the like. For example, a user device having a user interface platform for processing/analyzing tumor fraction data may communicate with another user device with the same platform, a regular user device without the same platform (e.g., a regular smartphone), a remote server, a physical device of a remote IoT local network, a wearable device, a user device communicably connected to a remote server, and etc.

The functional modules described herein are provided by way of example. It will be understood that different functional modules may be combined to create different utilities. It will also be understood that additional functional modules or sub-modules may be created to implement a certain utility.

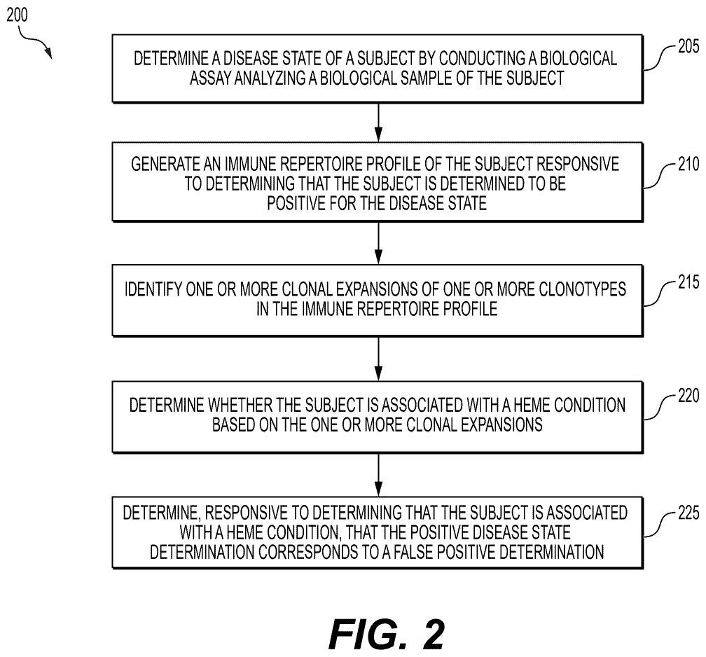

Referring now to FIG. 2, an exemplary workflow 200 is provided for determining whether a positive disease state determination for a subject is resultant from the presence of a heme condition. Aspects of the exemplary workflow 200 may be performed in accordance with some or all components described in FIGS. 1A and 1B.

At step 205, a disease state of a subject may be determined by conducting one or more biological assays analyzing a biological sample of the subject. In an aspect, biological samples, e.g., gDNA samples, cfDNA samples, etc., may be collected using minimally invasive methods, such as blood draws or other plasma collection procedures commonly used to obtain gDNA or cfDNA. That said, any suitable method of sample collection and any suitable sample type may be collected at step 205. In an aspect, a single sample may be collected from a subject or, alternatively, multiple samples may be collected from the subject (e.g., multiple samples may be collected from the subject at a single time point, multiple samples may be collected from the subject across two or more different time points, etc.). Further, step 205 may not include active sample collection and may instead refer to receipt of samples and/or data associated with samples that were previously collected. In one aspect, metadata associated with each sample may be collected, e.g., subject demographics, medical history, treatment regimens, and/or any relevant clinical information. This metadata may in some aspects provide context for interpreting methylation patterns and understanding the impact of cancer treatment on these patterns.

In an aspect, the collected biological samples may be analyzed via the performance of one or more biological assays. For example, methylation analysis may be conducted on the collected sample via a methylation sequencing process. It is important to note that although the workflow in FIG. 2 is described with reference to a cfDNA methylation-based assay that performs a multi-cancer test, such an assay is not limiting and another type of assay, e.g., another type of cfDNA assay, may be utilized to initially determine the disease state of an individual.

In an aspect, methylation analysis may involve the assessment of DNA methylation patterns at specific genomic regions, for example, at cytosine-phosphate-guanine (CpG) sites. In an aspect, various high-throughput technologies may be employed for methylation profiling, including one or more of bisulfite sequencing, methylated DNA immunoprecipitation sequencing (MeDIP-seq), DNA methylation microarrays, and the like. For simplicity purposes, bisulfite sequencing is the methylation profiling technique described herein, however, this designation is not intended to be limiting.

In an aspect, bisulfite sequencing may involve the treatment of DNA with sodium bisulfite, which converts unmethylated cytosines (C) into uracils (U) while leaving methylated cytosines unchanged. After bisulfite treatment, the DNA may be subjected to high-throughput sequencing, such as next-generation sequencing (NGS), to determine the methylation status of individual CpG sites across the genome. Whole-genome bisulfite sequencing (WGBS) provides comprehensive coverage of CpG sites and allows for a detailed assessment of methylation patterns. The generated methylation data may undergo bioinformatics analysis. In this regard, the methylation data may first undergo one or more preprocessing steps to ensure the quality and integrity of the methylation data. These steps may include one or more of: data cleaning, quality control, and the removal of artifacts or outliers that may affect the accuracy of the analysis. Preprocessing may also involve the alignment of sequence reads to a reference genome. The ratio of C to T at each CpG site may be used to calculate the methylation level.

In an aspect, the methylation level at each CpG site may be represented by a beta value, which are typically reported as decimal values ranging from 0 to 1. A beta value of 0 indicates that the CpG site is completely unmethylated. A beta value of 1.0 indicates that the CpG site is completely methylated. A beta value of 0.50 indicates that the CpG site is 50% methylated. Beta values offer a straightforward interpretation of DNA methylation levels. For example, a beta value of 0.2 at a specific CpG site suggests that 20% of the DNA molecules at the site are methylated, while the remaining 80% are unmethylated.

The computed beta values may be used in differentially methylated region (DMR) analysis to compare methylation levels between groups and determine statistically significant differences. More particularly, DMRs are genomic regions that exhibit differential methylation patterns between different groups or conditions. These regions are identified based on the differential methylation patterns observed across multiple CpG sites within a genomic region and are defined based on statistical comparisons of beta values between different groups or conditions. Accordingly, DMR analysis involves comparing beta values between groups to identify regions with differential methylation. Various tests, such as t-tests, nonparametric tests, or linear regression models, can be used to assess the significance of methylation differences at individual CpG sites or regions. DMRs may be defined based on statistical thresholds, such as p-values or adjusted p-values, indicating significant differences in methylation levels between groups.

In an aspect, data processing module 140 may be configured to transforming the sequencing data into a consistent and suitable format for training one or more machine learning models. The various steps involved in data preprocessing may include data cleaning (e.g., removal of any duplicate, incomplete, or erroneous entries from the dataset), missing value handling (e.g., resolving missing data points by employing appropriate techniques to estimate or fill in the missing values), normalization or standardization (e.g., rescaling the data to bring it to a common scale or distribution, which enables fair comparisons and prevents certain features from dominating the analysis due to their scales), and feature encoding (e.g., converting categorical variables into a numerical or binary representation that is suitable for machine learning models). It is important to note that the data preprocessing steps listed above may vary based upon the type of data collected and/or by the type of machine learning model that will be trained on the preprocessed data.

The preprocessed data may be passed to a feature selection component (not illustrated) of the data processing module 140 to identify and select the most relevant features from the cumulative dataset for model training. The feature selection process may reduce the dimensionality of the dataset by eliminating irrelevant or redundant features, which ultimately may improve model performance, facilitate faster model training and inference (i.e., working with a reduced set of features may reduce the computational complexity of training and inference processes), and contribute to enhanced model interpretation. In the context of the methylation approaches described herein, feature selection may involve the identification of a subset of DMRs, alongside individual CpG site beta values, that may be more relevant to the prediction of an individual's survival status. By incorporating both DMRs and beta values as features in the training and prediction process, the model may leverage the information from different scales of methylation data. DMRs capture larger-scale methylation patterns associated with specific genomic regions, whereas beta values provide detailed information about methylation levels at individual CpG sites. This combined approach allows for a comprehensive analysis of the methylation data and may improve the model's ability to capture the complexity and heterogeneity of methylation patterns associated with different outcomes.

In an aspect, one approach that may be leveraged to perform feature selection may be principal component analysis (PCA), which is a statistical technique that may be applied to the preprocessed data to capture the most informative features. More particularly, high-dimensional datasets, such as those generated from methylation assays, may contain a large number of features (e.g., methylation beta-values) that can be computationally demanding and may suffer from issues like overfitting, which occurs when a model performs well on the training data but fails to generalize to new data. PCA may transform the original dataset into a new set of uncorrelated variables called principal components (PCs), which are linear combinations of the original features. By capturing the maximum variance in the data, PCA may allow for dimensionality reduction while retaining the most relevant information. After computing the PCs, PCA may allow for dimensionality reduction by selecting a subset of the components that capture the most relevant information, which may be achieved by retaining the top PCs that explain a significant portion of the total variance in the data. The reduced feature set obtained from PCA may replace the original high-dimensional feature set in subsequent steps, such as in building a cancer classifier, as further described below.

The lower dimensionality data containing the selected features may be utilized as training data to train one or more machine learning models. In general, model training may correspond to teaching a classifier to recognize patterns and correlations between DMRs in a sample and those associated with known disease types. In an aspect, systems 100, 110 may include instructions for retrieving output features, e.g., based on the input of the machine learning models, and/or operating the displays contained in input and output module 120 to generate one or more output features. In some aspects, a system or device other than computer systems 100, 110 may be used to generate and/or train the machine learning models. For example, such a system may include instructions for generating the machine learning model, the training data and/or ground truth, and/or instructions for training the machine learning model. A resulting trained machine learning model may then be provided to the computer systems 100, 110.

In some aspects, the machine learning model may be constructed using supervised learning, e.g., where a ground truth is known for the training data provided. The training may proceed by feeding a sample of training data into a model with variables set at initialized values. The model learns to capture the relationships between the input features and the corresponding target variable. For example, the model may be trained to identify a correlation between a specific methylation pattern (e.g., as represented by X principal components) of a reference subject and the corresponding disease state label that the subject is associated with.

Although a variety of different types of machine learning architectures may be employed (e.g., convolutional neural networks, support-vector machines, gradient boosting machines, etc.), as a non-limiting designation, the type of machine learning architecture referenced throughout the remainder of this disclosure is a random forest classifier. A random forest is an ensemble learning method that combines multiple decision trees to make predictions. Each decision tree in the random forest is trained on a subset of the training data and a subset of the features. At each split within each tree, a subset of the CpG site beta values and/or DMR features are randomly selected for consideration. The random forest algorithm aggregates the predictions of all the individual trees to make the final prediction. The random forest classifier may therefore utilize the training dataset with the labeled disease states to learn the relationship between certain methylation patterns and the corresponding disease states they may be associated with. Through this training, the random forest classifier may be trained to predict whether a subject is likely to test positive for a specific disease state based on the methylation patterns captured by the selected features. Once the random forest classifier is trained, it can be used to make predictions on new, unseen samples.

After a model is constructed and trained, a validation process may be implemented to check its performance. More particularly, the classifier may make predictions on a testing set. The predicted outcomes on the testing set may be compared to the known outcomes (ground truth) to evaluate the performance of the classifier. More particularly, the classifier may be evaluated using appropriate metrics, such as area under the receiver operating characteristic curve (AUC-ROC), to assess the model's predictive capabilities. The AUC-ROC is a metric for classification accuracy of a binary predictive model across all score cutoffs. It measures a curve for all values of apparent true positive rates for equivalent false positive rates. It may vary from 0.5, indicating predictions are effectively random and the model has no predictive value, up to 1, indicating a perfectly predictive classifier.

To obtain a more robust assessment of the model's performance, a cross-validation evaluation technique may be employed to estimate the performance of the classifier on unseen data. Such a process may first involve dividing the available dataset into X equal-sized subsets, or folds, generally known as “K-folds.” Each fold contains a roughly equal distribution of samples across the different classes or outcomes. The cross-validation process involves “K” iterations, where each iteration uses K−1 folds for training and the remaining fold for testing. More particularly, one of the folds for each iteration is treated as the testing set, while the other K−1 folds are combined to form the training set. In each iteration, the model is trained on the training set using the chosen algorithm and hyperparameters. The trained model is then used to predict the outcomes of the samples in the testing fold. The predicted outcomes are compared to the known outcomes (ground truth) to evaluate the model's performance. The performance metrics obtained from each iteration (e.g., accuracy, precision, recall, etc.) are collected and the aggregated results provide an estimate of the model's performance across multiple test sets. These results help assess how well the model generalizes to unseen data and may provide a more reliable evaluation compared to a single train-test split, as described above.

In an aspect, cross-validation, e.g., nested cross-validation, may also be used for hyperparameter tuning. Different combinations of hyperparameters may be evaluated using cross-validation, and the set of hyperparameters that yield the best performance may be selected. For example, data analysis module 150 may be configured to iterate over different hyperparameter settings (e.g., maximum depth, number of trees) and evaluate the model's performance using cross-validation. The hyperparameter configuration that yields the best average performance (e.g., the highest AUC-ROC score, etc.) across the iterations may be selected as the “champion” or “optimal” hyperparameter configuration set.

In an aspect, a fully trained and validated model may then be leveraged to predict whether a test sample associated with a subject is positive for a particular disease state, e.g., a type of cancer. More particularly, the classifier may be configured to provide a binary indication of whether the subject likely contains, or does not contain, the disease state. In another aspect, the classifier may be configured to generate a score that is representative of a disease state likelihood of a subject (e.g., a higher score represents a higher likelihood of the subject having the disease state, etc.).

In an aspect, steps 210-225 are representative of a companion test that may be used in conjunction with the methylation-based multi-cancer test described with respect to step 205. The companion test aims to quantify abnormal clonal lymphocytes or methylation signatures present in a sample (e.g., a WBC sample) derived from individuals who have undergone the primary cfDNA methylation-based multi-cancer test, as described above. This companion test further provides an indication or probability that a participant with a positive disease test may harbor a precursor blood condition, and the positive signal is not indicative of cancer. In an aspect, the companion test may be performed before, during, or after the performance of the cancer assay.

At step 210, the systems 100, 110 may be configured to generate an immune repertoire profile of the subject responsive to determining that the subject has tested positive for a particular disease state. The immune repertoire profile provides an analysis of the diversity and composition of lymphocytes (e.g., T and B cells) in an individual's immune system. It can provide insights into the immune system's ability to recognize and respond to various antigens, including those associated with precursor blood conditions or cancer. Changes in the immune repertoire, such as through clonal expansion of specific lymphocyte populations, may indicate underlying health conditions or abnormalities. In an aspect, the immune repertoire profile may be generated from an immune repertoire sequencing of the biological sample, which enables the identification and quantification of different T and B cell clones based on the unique sequences of their antigen receptors. Through immune repertoire sequencing, a multitude of clonotypes and their corresponding clonal frequencies within the immune system may be captured.

In an aspect, immune repertoire sequencing may first involve sample collection (e.g., blood) from the subject, or the receipt of a sample previously taken from the subject. Genomic DNA may then be extracted from the collected sample and sequenced (e.g., using a high-throughput sequencing technique). The raw sequencing data may be processed to identify and annotate the unique sequences of T and B cell receptors. Clonotypes, representing distinct T or B cell clones with unique receptor sequences, may be identified. The number of occurrences of each unique clonotype sequence in the sequencing data may be counted, wherein the count represents the raw abundance of each clonotype. The raw counts may be normalized to account for variations in sequencing depth. This normalization ensures that the clonal frequency calculation is not biased by differences in the total number of sequencing reads between samples. In an aspect, the clonal frequency of a specific clonotype may be calculated as the ratio of the normalized count of that clonotype to the total number of normalized counts for all clonotypes in the sample. This calculation results in a percentage that represents the proportion of the total immune repertoire made up by that particular clonotype.

At step 215, one or more clonal expansions of one or more clonotypes in the immune repertoire profile may be identified. Clonal expansions may signify an abnormal increase in the abundance of specific lymphocyte populations, and their detection may be important to better understanding potential health conditions. More particularly, clonal expansions may be indicative of various conditions, including hematologic malignancies or precursor conditions.

In an aspect, the identification of clonal expansions may involve analyzing the sequencing data to recognize instances where certain T or B cell clones are overrepresented or expanded in comparison to the normal, diverse repertoire profile. In this regard, the identified clonotypes and their frequencies may be compared to what would be expected in a normal, diverse immune repertoire. A deviation from the expected diversity may indicate clonal expansion. In an aspect, a threshold may be established to define what constitutes a clonal expansion. This threshold may be determined based on statistical analysis or established norms for the specific population or condition being studied/tested for. Clonal expansion is considered to occur when certain clonotypes surpass the defined threshold, thereby suggesting an abnormal increase in their abundance compared to the baseline.

At step 220, based on the identified clonal expansions within the immune repertoire profile, a determination may be made about whether the subject is associated with a heme condition. The presence of clonal expansions in the immune repertoire, particularly those associated with hematologic malignancies, may provide insights into the subject's health status. Examples of heme conditions may include, e.g., one or more of a premalignant/precursor heme condition, a malignant/cancerous heme condition, Monoclonal B-cell lymphocytosis (MBL), or Monoclonal gammopathy of undetermined significance (MGUS).

In an aspect, the identified clonal expansions may be assessed for their association with known patterns or signatures that may be indicative of heme conditions. This assessment may involve comparing the observed clonal expansions to established databases or literature that link specific clonotypes to hematologic malignancies or precursor conditions. Specific clonal expansion patterns, such as the presence of certain immunoglobulin rearrangement or T cell receptor sequences, may be indicative of particular heme conditions. In an aspect, certain thresholds may be established to define the significance of the observed clonal expansions in relation to heme conditions. More particularly, a baseline or reference data set representing the expected distribution of clonal frequencies in a healthy population may be established or referenced. This baseline may be derived from a control group or established databases of immune repertoire data from individuals without heme conditions. Statistical analysis (e.g., calculation of mean, median, standard deviation, etc.) may be calculated for the clonal frequencies of identified expansions and the overall immune repertoire. In an aspect, clonal expansion patterns and/or the expected baseline distributions may be stored on database 20, 130 and the analysis processing may be performed using one or both of data analysis or processing modules 140, 150.

Additionally or alternatively to the foregoing, a trained machine learning model may be employed to identify patterns in the clonal expansion data and classify subjects into different groups, including those associated with heme conditions. More particularly, in some cases, the companion test may utilize machine learning to determine whether the heme condition is present. For instance, the companion test may utilize a binary and/or multiclass classifier that is trained by inputting sets of training samples with their feature vectors into the classifier and adjusting classification parameters so that a function of the classifier accurately relates the training feature vectors to their corresponding label. The training samples may be grouped (e.g., by an analytics system) into sets of one or more training samples for iterative batch training of the classifier. After inputting all sets of training samples including their training feature vectors and adjusting the classification parameters, the classifier can be sufficiently trained to label test samples according to their feature vector within some margin of error. The analytics system can train the classifier according to any one of a number of methods. As an example, the binary classifier may be a L2-regularized logistic regression classifier that is trained using a log-loss function. As another example, the classifier can be a multinomial logistic regression. In practice, either type of classifier can be trained using other techniques. These techniques are numerous, including potential use of kernel methods, random forest classifier, a mixture model, an autoencoder model, machine learning algorithms such as multilayer neural networks, etc. In some examples, the classifier can include a logistic regression algorithm, a neural network algorithm, a support vector machine algorithm, a Naïve Bayes algorithm, a nearest neighbor algorithm, a boosted trees algorithm, a random forest algorithm, a decision tree algorithm, a multinomial logistic regression algorithm, a linear model, or a linear regression algorithm.

In view of the foregoing, a machine learning model may be iteratively trained based on training samples. The training samples may contain immune repertoire profiles of reference individuals with known disease states. For instance, the training sample disease states may include a first disease state where no heme condition is diagnosed, a second disease state where a heme condition is diagnosed, a third disease state where a cancer is diagnosed, and/or a fourth disease state where no cancer is diagnosed. In an aspect, the reference individuals in the training samples may include those individuals having at least the foregoing types of disease states.

In an aspect, the machine learning model may be associated with a plurality of weight coefficients that may be applied during iterative model training. More particularly, data associated with the training samples may first be provided to the model. The model may then generate predictions of disease states for each of the training samples. The predicted disease states may then be compared to the actual disease states of the reference individuals from whom the training samples were acquired and, thereafter, certain weight coefficients of the model may be adjusted based on this comparison. In an aspect, the prediction generation may be facilitated via forward propagation, and the weight coefficient adjustment may be facilitated via back propagation. In an aspect, the weight coefficients may be adjusted using coordinate descent.

At step 225, the method may comprise determining, based on the determined heme condition, whether a positive disease state determination by the disease state classifier is a false positive. In an aspect, if the identified heme-associated clonotypes in the immune repertoire profile are found to be the primary, or significant, contributors to the positive disease state determination from the disease-state assay, and there is no evidence of a true pathological condition, the positive disease state may be deemed a false positive. In another aspect, a determination may be made that the positive disease state determined by the disease state classifier was not a false positive, but that at least a subset of the identified heme-associated clonotypes in the immune repertoire profile generated confounding information that affected a degree of the disease state determination. In an aspect, these determinations may be facilitated by the same or different trained machine learning model as previously described above. Additionally or alternatively, the identified heme-associated clonotypes may be cross-referenced with the results from other biological assays that assess the disease state. This may involve examining for patterns of correlation or discordance between the immune repertoire data and other diagnostic information, a process which may be conducted automatically by computer systems 100, 110 or performed manually by the user.

### Objectives

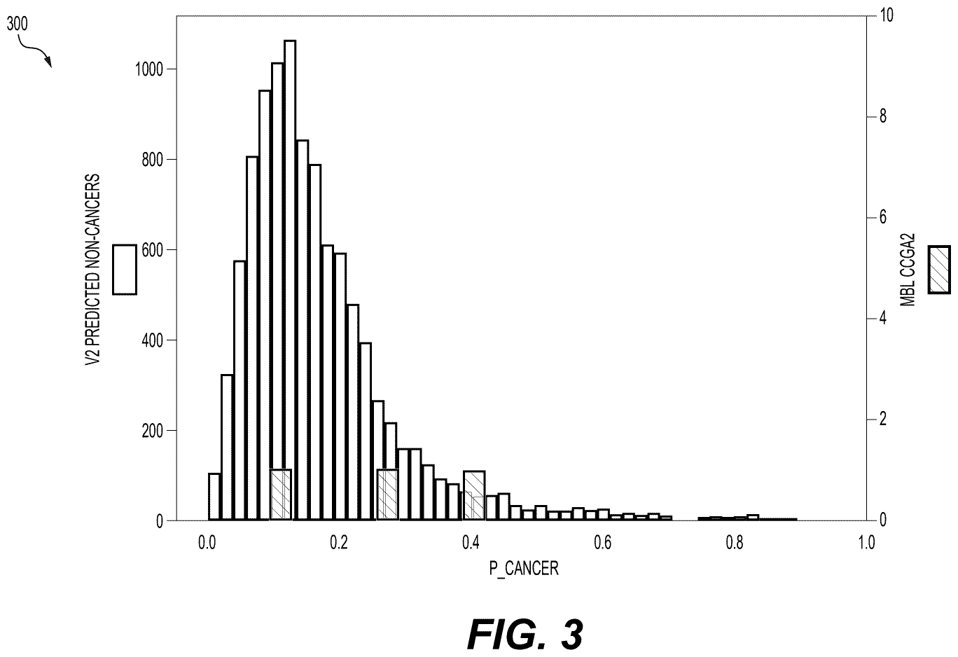

The concepts described herein were developed in furtherance of observations made on available data. FIGS. 3-10 provide underlying support for the concept that determining the heme condition of a subject may promote accurate detection of a different disease state (e.g., cancer) in the subject. For instance, graph 300 in FIG. 3 presents cancer score data associated with samples of subjects known to have the precursor heme condition MBL. Although the MBL scores in graph 300 did not trigger a positive finding from the relevant classifier, MBL cases have been known to be detected as false positives for cancer detection, and the identification of the threshold clonal expansion required to trigger a positive identification from a classifier may be a relevant metric.

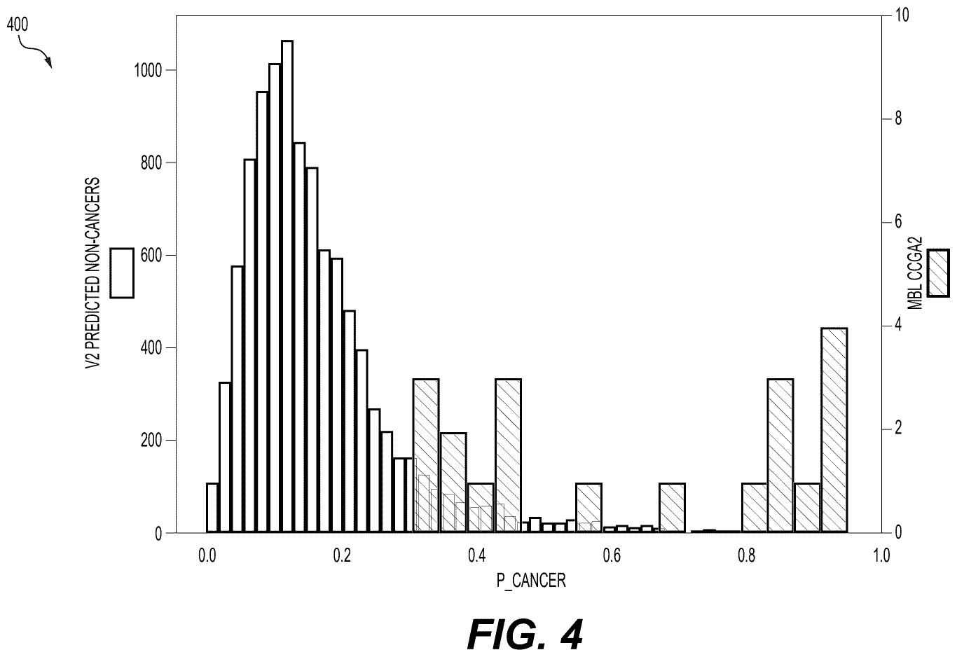

Graph 400 in FIG. 4 presents additional cancer score data associated with samples of subjects known to have the precursor heme condition MGUS. The MGUS cancer scores present in graph 400 are associated with those samples having higher scores that are likely to result in a false positive determination around the decision boundary.

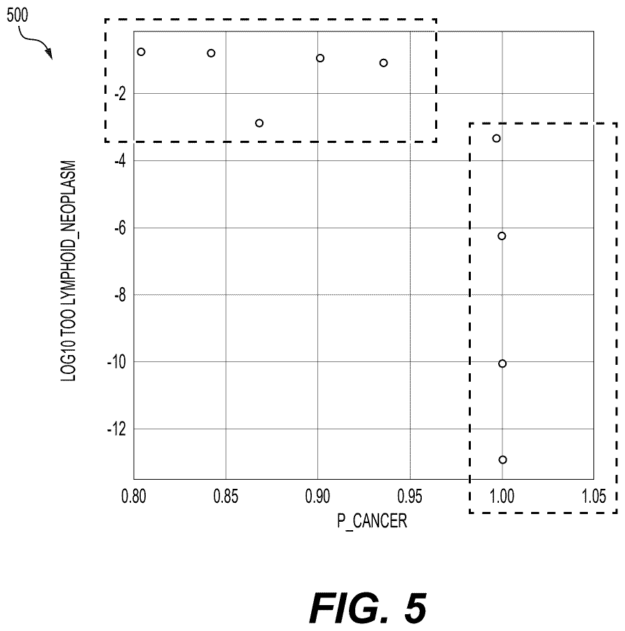

Graph 500 in FIG. 5 presents data associated with solid cancer false positives. More particularly, a subset of the data points in FIG. 5 presented with lower cancer signal but higher heme at the tissue of origin (TOO). These samples may originate from the upper GI, pancreas, gallbladder, colon, breast, or prostate. Another subset of data points in FIG. 5 present with high cancer signal but lower heme at the TOO. These samples may originate from the kidney, lung, ovary, colon, and/or liver.

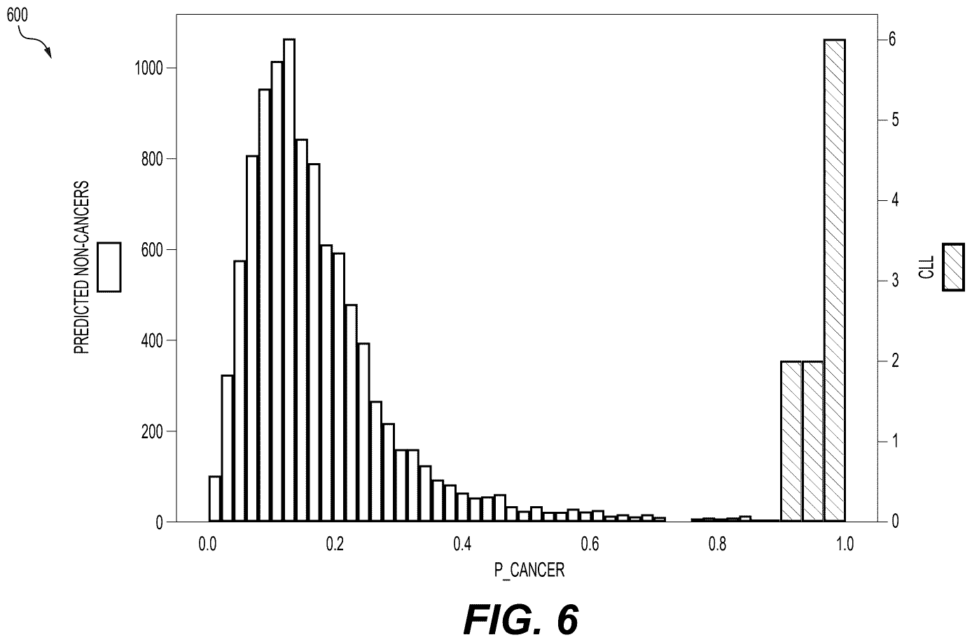

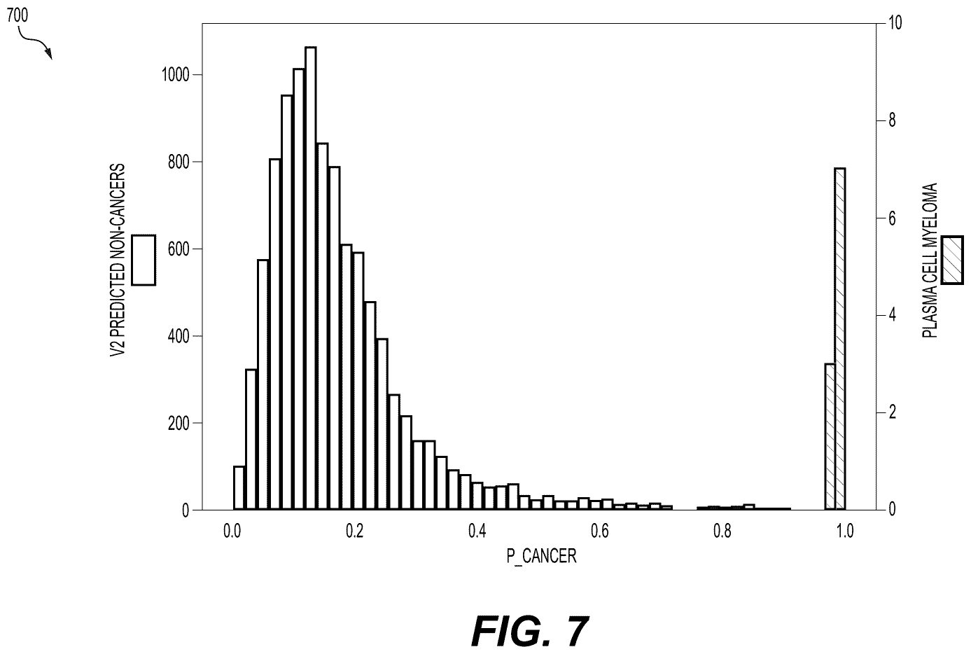

Graph 600 in FIG. 6 provides data associated with a group of participants who had leukemia with a heme subtype of CLL and who also had WBC sequencing data available. Graph 700 in FIG. 7 provides data associated with a group of participants who had MM with a heme subtype of plasma cell myeloma. Graphs 600 and 700 collectively present data that may aid in the assessment of assay LOD.

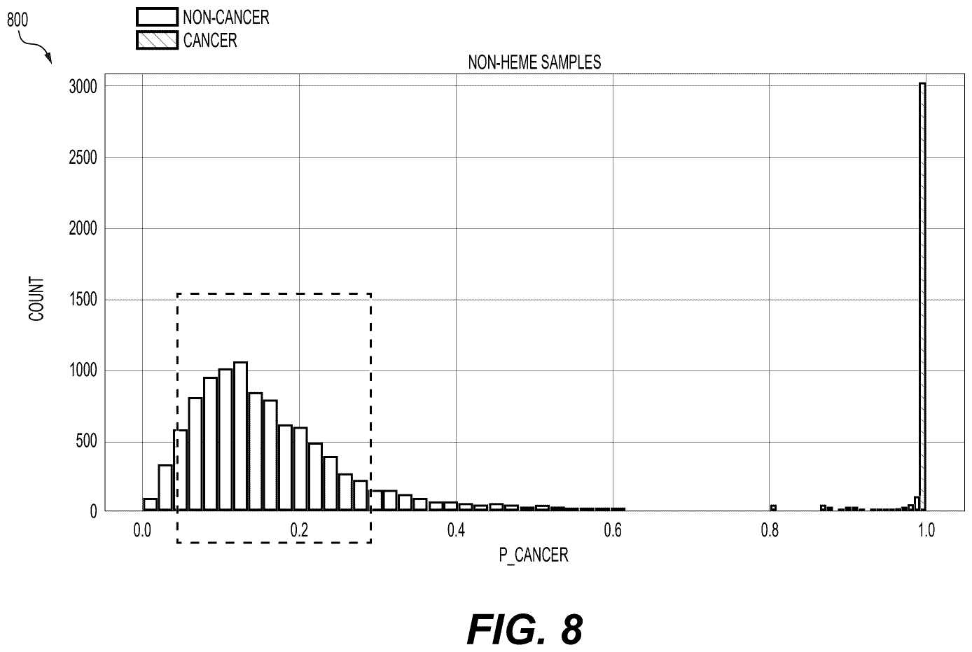

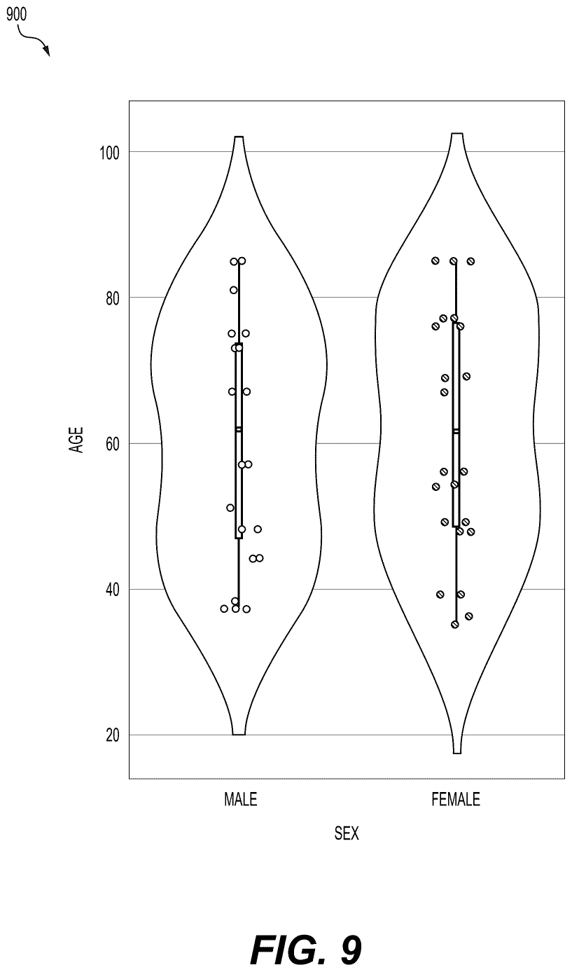

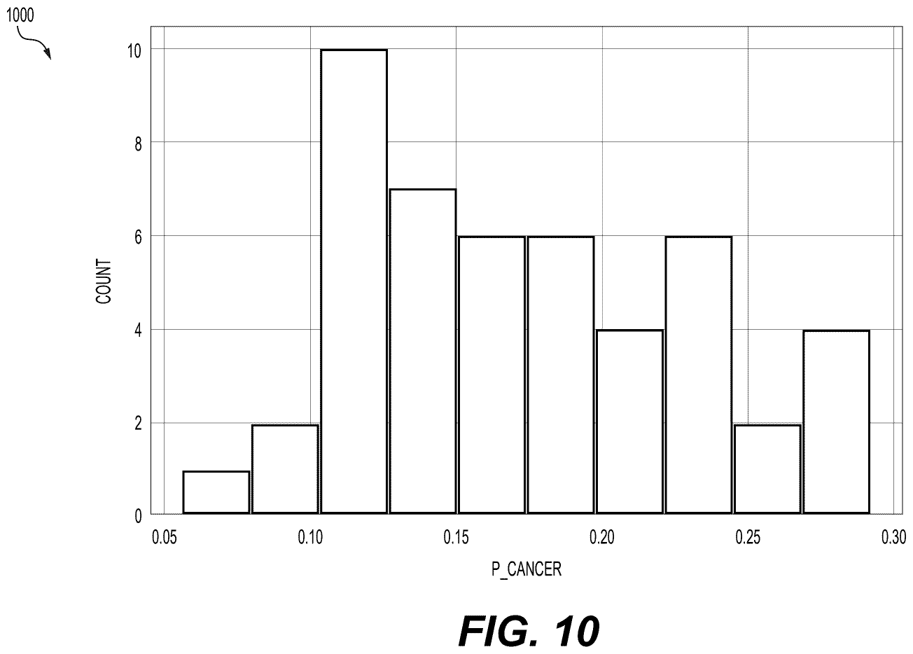

Graph 800 in FIG. 8 presents data associated with a distribution of the negative controls that were selected. More particularly, graph 800 illustrates the clonality distribution of normal non-cancer participants. Diagram 900 in FIG. 9 indicates that the participants in the negative controls represented in FIG. 8 were balanced by age and sex. Graph 1000 in FIG. 10 provides a histogram of the cancer scores of the participants from FIG. 8.

### Experimental Results

In an aspect, the embodiments described herein were practically tested. Using LymphoTrack IGH FR1 Assay (Invivoscribe, Inc), DNA was sequenced that was derived from WBCs from a subset of enrollees in a study comprising non-cancer (NC) participants, balanced for age and gender, and participants diagnosed with a hematological precursor and neoplastic conditions (HPNC) (e.g., chronic lymphocytic leukemia (CLL), multiple myeloma MM, MBL, or MGUS). Additional samples were titrated and processed to determine the limit of quantification (LoQ).

Using 12 titration samples, the LoQ of detecting an unknown clone against a polyclonal background was determined to be 0.1% of the total reads. The threshold used to determine evidence of CE was 2.5% of the total reads. Samples were sequenced from 112 individuals, 67 of whom were NC participants (35 male of median [range] age 67 [33-85] years; 32 female of 60 [30-85] years). Most NC samples (57/67; 85%) lacked evidence of CE: median top clone percent total reads (PTR) of 0.10% (mean: 0.18%; SD: 0.24%) and median Simpson clonality of 0.0021 (mean: 0.0030; SD: 0.0030). The remaining samples (10/67; 15%) showed evidence of CE: 9/10 (90%) were monoclonal with clonal PTR ranging from 2.7% to 80.4% (median: 6.0%), and 7/9 had a mutation rate >2%, indicating somatic hypermutation (SHM). One sample exhibited oligoclonality with 2 clones (6.6% and 3.2%), and both had SHM. A positive correlation between the top clone PTR and age (=0.41) was also observed. All CLL samples (9/11 monoclonal; 2/11 oligoclonal) and 3/4 MBL samples showed evidence of CE. Consistent with the observation that a limited number of plasma cells are expected to be in circulation, only 3/10 MM samples and 7/20 MGUS samples showed evidence of CE; 3 MGUS samples had a top clone PTR of >40%.

Based on this data, while most NCs did not show evidence of CE, 15% of them had evidence of expanded lymphoid clones with SHM. The PTR of the top clone underscores the higher frequency of CE associated with increased age in asymptomatic participants. Clonal expansion of blood cell lineages can also carry genetic or epigenetic alterations that are similar to those associated with cancer. Thus, differentiating aberrant signals originating from various hematologic compartments may be for accurate early cancer detection.

In general, any process discussed in this disclosure that is understood to be computer-implementable may be performed by one or more processors of a computer system, such as system environment 110, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable types of processing unit.

A computer system, such as system environment 110, may include one or more computing devices. If the one or more processors of the computer system are implemented as a plurality of processors, the plurality of processors may be included in a single computing device or distributed among a plurality of computing devices. If a system environment comprises a plurality of computing devices, the memory of the computer system may include the respective memory of each computing device of the plurality of computing devices.

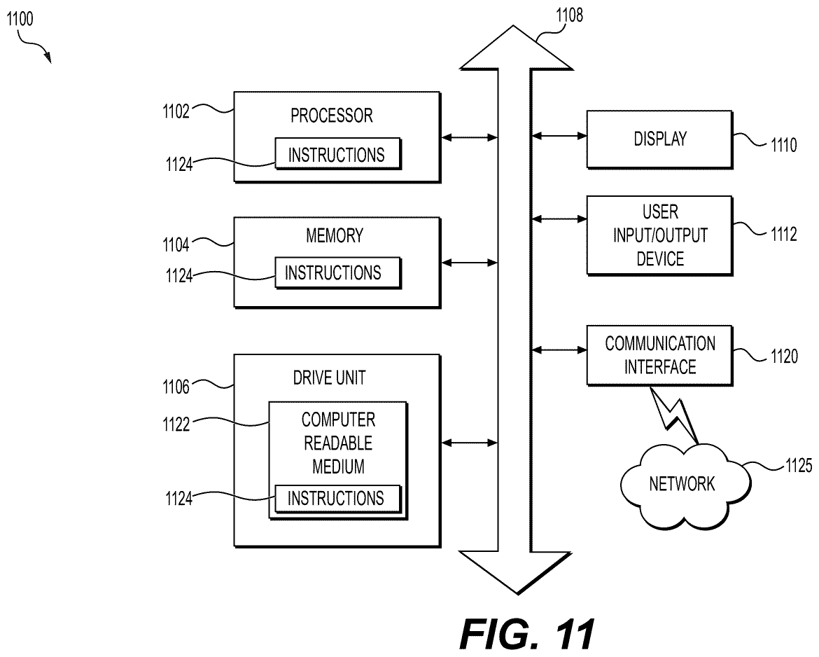

FIG. 11 is a simplified functional block diagram of a computer system 1100 that may be configured as a computing device for executing the processes described herein, according to exemplary embodiments of the present disclosure. FIG. 11 is a simplified functional block diagram of a computer that may be configured according to exemplary embodiments of the present disclosure. In various embodiments, any of the systems herein may be an assembly of hardware including, for example, a data communication interface 1120 for packet data communication. The platform also may include a central processing unit (“CPU”) 1102, in the form of one or more processors, for executing program instructions. The platform may include an internal communication bus 1108, and a storage unit 1106 (such as ROM, HDD, SDD, etc.) that may store data on a computer readable medium 1122, although the system 1100 may receive programming and data via network communications via electronic network 1125 (e.g., voice, video, audio, images, or any other data over the electronic network 1125). The system 1100 may also have a memory 1104 (such as RAM) storing instructions 1124 for executing techniques presented herein, although the instructions 1124 may be stored temporarily or permanently within other modules of system 1100 (e.g., processor 1102 and/or computer readable medium 1122). The system 1100 also may include input and output ports 1112 and/or a display 1110 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of +10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only if the alternatives are mutually exclusive, although the disclosure supports a definition that refers to only alternatives and “and/or.” As used herein “another” may mean at least a second or more.

As used herein, the term “user” generally encompasses any person or entity, such as a researcher and/or a care provider (e.g., a doctor, etc.), who may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

1.-43. (canceled)

44. A method for determining a heme condition of a subject from a biological sample of the subject, the method comprising:
receiving an immune repertoire profile of the subject, the immune repertoire profile generated from an immune repertoire sequencing of deoxyribonucleic acid (DNA) in the biological sample and comprising a plurality of clonotypes of the DNA and corresponding clonal frequencies of the clonotypes;
identifying one or more clonal expansions of one or more clonotypes in the immune repertoire profile;
inputting the clonal frequencies associated with the one or more clonal expansions to a machine learning model that is iteratively trained based on training samples, the training samples comprising immune repertoire profiles of reference individuals with known disease states, the disease states comprising a first disease state where no heme condition is diagnosed, a second disease state where the heme condition is diagnosed, and a third disease state where a cancer is diagnosed, wherein the reference individuals in the training samples comprise individuals with one or more of the disease states; and
generating, using the machine learning model, a determination of the heme condition of the subject.

45. The method of claim 44, wherein the heme condition is Monoclonal B-cell lymphocytosis (MBL).

46. The method of claim 44, wherein the heme condition is Monoclonal gammopathy of undetermined significance (MGUS).

47. The method of claim 44, wherein the biological sample comprises a plasma sample and the DNA comprises cell-free deoxyribonucleic acid (cfDNA).

48. The method of claim 44, wherein the method is a companion test of a cancer test and wherein the cancer test includes a methylation sequencing of a cell-free deoxyribonucleic acid (cfDNA) sample of the subject.

49. The method of claim 44, wherein the subject is previously determined to have cancer via a cancer test, and wherein the determination that the subject has the heme condition indicates that the cancer determination is a false positive.

50. The method of claim 44, wherein the determination identifies whether the subject has any heme condition and/or a type of heme condition of the subject.

51. The method of claim 44, wherein the machine learning model is trained by a supervised learning technique, and wherein at least one of the training samples uses the disease state of the individual as a training label in the supervised learning technique.

52. The method of claim 44, wherein the machine learning model is associated with a plurality of weight coefficients, and iteratively training of the machine learning model comprises:
inputting data associated with the training samples to the machine learning model;
generating, using the machine learning model, predicted diseases states of the training samples;
comparing the predicted diseases states to the disease states of the individuals; and
adjusting the weight coefficients of the machine learning model based on comparing the predicted diseases states to the disease states of the individuals.

53. The method of claim 52, wherein generating the predicted diseases states of the training samples is performed in forward propagation of the training of the machine learning model and adjusting the weight coefficients of the machine learning model is performed in back propagation of the training of the machine learning.

54. A system comprising:
one or more processors;
one or more computer readable media storing instructions that are executable by the one or more processors to perform operations to:
receive an immune repertoire profile of the subject, the immune repertoire profile generated from an immune repertoire sequencing of deoxyribonucleic acid (DNA) in the biological sample and comprising a plurality of clonotypes of the DNA and corresponding clonal frequencies of the clonotypes;
identify one or more clonal expansions of one or more clonotypes in the immune repertoire profile;
input the clonal frequencies associated with the one or more clonal expansions to a machine learning model that is iteratively trained based on training samples, the training samples comprising immune repertoire profiles of reference individuals with known disease states, the disease states comprising a first disease state where no heme condition is diagnosed, a second disease state where the heme condition is diagnosed, and a third disease state where a cancer is diagnosed, wherein the reference individuals in the training samples comprise individuals with one or more of the disease states; and
generate, using the machine learning model, a determination of the heme condition of the subject

55. The system of claim 54, wherein the heme condition is Monoclonal B-cell lymphocytosis (MBL).

56. The system of claim 54, wherein the heme condition is Monoclonal gammopathy of undetermined significance (MGUS).

57. The system of claim 54, wherein the system is a companion test of a cancer test and wherein the cancer test includes a methylation sequencing of a cell-free deoxyribonucleic acid (cfDNA) sample of the subject wherein the cancer test includes a methylation sequencing of a cell-free deoxyribonucleic acid (cfDNA) sample of the subject.

58. The system of claim 54, wherein the subject is previously determined to have cancer via a cancer test, and wherein the determination that the subject has the heme condition indicates that the cancer determination is a false positive.

59. The system of claim 54, wherein the determination identifies whether the subject has any heme condition and/or a type of heme condition of the subject.

60. The system of claim 54, wherein the machine learning model is trained by a supervised learning technique, and wherein at least one of the training samples uses the disease state of the individual as a training label in the supervised learning technique.

61. The system of claim 54, wherein the machine learning model is associated with a plurality of weight coefficients, and iteratively training of the machine learning model comprises:
inputting data associated with the training samples to the machine learning model;
generating, using the machine learning model, predicted diseases states of the training samples;
comparing the predicted diseases states to the disease states of the individuals; and
adjusting the weight coefficients of the machine learning model based on comparing the predicted diseases states to the disease states of the individuals.

62. The system of claim 61, wherein generating the predicted diseases states of the training samples is performed in forward propagation of the training of the machine learning model and adjusting the weight coefficients of the machine learning model is performed in back propagation of the training of the machine learning.

63. A non-transitory computer-readable medium storing computer-executable instructions which, when executed by a system, cause the system to perform operations comprising:
receiving an immune repertoire profile of the subject, the immune repertoire profile generated from an immune repertoire sequencing of deoxyribonucleic acid (DNA) in the biological sample and comprising a plurality of clonotypes of the DNA and corresponding clonal frequencies of the clonotypes;
identifying one or more clonal expansions of one or more clonotypes in the immune repertoire profile;
inputting the clonal frequencies associated with the one or more clonal expansions to a machine learning model that is iteratively trained based on training samples, the training samples comprising immune repertoire profiles of reference individuals with known disease states, the disease states comprising a first disease state where no heme condition is diagnosed, a second disease state where the heme condition is diagnosed, and a third disease state where a cancer is diagnosed, wherein the reference individuals in the training samples comprise individuals with one or more of the disease states; and
generating, using the machine learning model, a determination of the heme condition of the subject.
